(19) United States
(12) Patent Application Publication
(10) Pub. No.: US 2010/0317827 A1
(43) Pub. Date: Dec. 16, 2010

(54) Method of Purifying a Peptide

(75) Inventors: Nitesh Dave, Bangalore (IN); Krishnamurthy Venkatesan, Chennai (IN); Ramprabu Nagarajan, Trichy District (IN); Harish Iyer, Bangalore (IN)

Correspondence Address: HARNESS, DICKEY & PIERCE, P.L.C., P.O. BOX 828, BLOOMFIELD HILLS, MI, 48303, US

(73) Assignee: BIOCON LIMITED, Bangalore, Karnataka (IN)

(21) Appl. No.: 12/866,313

(22) PCT Filed: Mar. 26, 2008
(86) PCT No.: PCT/IN08/00191

§ 371 Date: Aug. 5, 2010

(30) Foreign Application Priority Data
Feb. 6, 2008 (IN) 00311/CHE/2008

(51) Int. Cl. C07K 14/435; C07K 1/18; C07K 2/00; C07K 7/06

(52) U.S. Cl. 530/324; 530/344; 530/300; 530/329

(57) ABSTRACT

The invention relates, interalia, to the field of purification of peptides, notably cyclic or non-cyclic peptides their analogs or derivatives thereof. More particularly, the invention relates to a simplified and optimized purification process of cyclic peptides from a composition comprising the said peptide and at least one related impurity by chromatographic procedures enabling high yields, selectivity and purity of the desired end product. The improved process is particularly useful for the preparation of eptifibatide, exenatide, atosiban, nesiritide and their respective derivatives and analogs. The polypeptides are prepared in high purity of at least about 96%, and preferably at least about 99%.

### FIELD OF THE INVENTION

The invention relates, interalia, to the field of purification of peptides, notably cyclic or non-cyclic peptides their analogs or derivatives thereof. More particularly, the invention relates to a simplified and optimized purification process of cyclic peptides from a composition comprising the said peptide and at least one related impurity by chromatographic procedures enabling high yields, selectivity and purity of the desired end product. The improved process is particularly useful for the preparation of eptifibatide, exenatide, atosiban, nesiritide and their respective derivatives and analogs. The polypeptides are prepared in high purity of at least about 96%, and preferably at least about 99%.

### BACKGROUND AND PRIOR ART OF THE INVENTION

A significant aspect of the production of recombinant (genetically engineered) peptides, including cyclic peptides, its derivatives and analogs thereof intended for therapeutic use in humans or animals is the purification process in question to obtain sufficiently high level of selectivity, productivity and purity, such that the desired protein is essentially free of contamination with extraneous proteins which may arise in the production process.

Various kinds of impurities are generated during peptide synthesis such as diastereomers, hydrolysis products of labile amide bonds, deletion sequences formed predominantly in solid phase peptide synthesis, insertion peptides and by-products such as polymorphic forms are formed during removal of protection groups in the final step of the synthesis. These are often some of the by-products associated with formation of cyclic peptides containing disulphide bonds [Bodansky et.al, Principles of Peptide Synthesis; Springer-Verlag; berlin, 1993]. There is a need in the art to develop a simple chromatographic purification procedure that may be operational on a large scale with minimal steps to isolate the desired peptide in the mother liquor from a complex mixture of related impurities. Essentially, most of the impurities generated during the synthesis process cannot be removed by a single chromatographic method, but rather by a combination of methods such as Reverse-phase chromatography and cation-exchange chromatography is to prepare the drug product, drug substance in formulation buffer as in context of the present invention.

The subject invention encompasses two operational chromatographic procedures including Reverse-phase chromatography and cation-exchange chromatography.

A number of different chromatographic procedures are applied to obtain the desired end result with respect to purity and yield. Reverse-phase chromatography is one of the most powerful methods of purification employed utilizing hydrophobic interactions as the main separation principle. Reverse phase liquid chromatography (“R^{P}-LC”) and reverse phase high-performance liquid chromatography (“RP-HPLC”) are commonly used to purify molecules such as peptides and proteins, produced by either synthetic or recombinant methods. RP-LC and RP-HPLC methods can efficiently separate closely related impurities and have been used to purify many diverse molecules (Lee et al., “Preparative HPLC,” 8^{th} Biotechnology Symposium, Pt. 1, 593-610 (1988)). Further, RP-LC and RP-HPLC have been successfully used to purify molecules, particularly; proteins on an industrial scale (Olsen et al., 1994, J. Chromatog. A, 675, 101).

The Ion exchange chromatography principle includes two different approaches: anion exchange and cation exchange according to the charge of the ligands on the ion exchange resin. A conventional IEC purification process usually consists of one or more: equilibration sections, application or loading sections, wash sections, elution sections, and regeneration sections (cf. Remington's Pharmaceutical Sciences, Gennaro, ed., Mack Publishing Co., Easton, Pa., 1990, or Remington: The Science and Practice of Pharamacy, 19th Edition (1995)).

Every attribute of the chromatographic procedure has a significant role to play in obtaining the desired protein product. Various chromatographic media have been used for large scale purifications of peptides on reversed phase resins. Among the most popular are those based on C-4, C-8 and C-18 alkyl chains attached to a silica surface. Some of the important considerations include shape and size of the resin particles of the stationary phase. Some of the other significant properties include the buffer system, the flow rate, pH etc. Despite the improvements for peptide purification, some purified peptides still contain undesired amounts of unacceptable counterions. Owing to this believed theory of operation, reference is made through the application to an alternate method of purification to overcome the problems faced in art.

US2006148699 relates to a method of RP-HPLC based purification of peptides comprising washing of the column with an aqueous solution of a pharmaceutically acceptable counterion salt; and eluting the peptide from the column with a solvent mixture of an organic solvent and an acid of a pharmaceutically acceptable counter-ion wherein the aqueous solution has a pH of at least about 6. Subsequent dependant claims over cyclic, non-cyclic peptides and eptifibatide also being specifically claimed.

WO2005100388 discloses RP-HPLC purification of exendin-4 using acetonitrile-water gradient and retrieving product with purity of 97.5%.

WO2005019262 relates to the use of polystyrene/divinylbenzene based resin for RP-HPLC purification of glucagon like peptides.

There is a need in art for an efficient chromatographic protocol for selectively separating molecules such as cyclic peptides post preparation by solid phase synthesis to obtain highly pure peptide end products. This need would be satisfied when the process duplicates as much as possible the yield, purity, throughput, and operating conditions of the chromatographic process wherein elution is conducted by selected solvent system, pH range and other related factors. Operating procedures may be advantageously employed for commercial separations.

Another important advantage of the separation process according to the present invention is that they may be scaled up in a reproducible and consistent manner. Further, the process of the present invention affords products which are superior to those obtained by purification methods hitherto known and give higher yields.

The present invention on the application of pH-buffered solvents comprising a polar solvent as the organic elution agents for RP-HPLC purification of cyclic peptides and analogs or derivatives thereof at pH in the range of 2-8. The present invention facilitates increased separation efficiency, higher purity and easy operation for industrial use compared to the current state of the art within RP-HPLC purification of cyclic peptides using aforesaid solvent systems. Surprisingly, separation of target cyclic peptide compounds and related impurities is improved by the new methodology employed in the present invention and results in more pure cyclic like peptide products.

The elution of peptide in formulation solution has advantages as compared to the conventional method. The conventional method comprises of freeze drying the purified peptide and redissolving the freeze dried powder in a solution prior to addition of excipients. The elution of peptide in formulation solution avoids the operation of freeze drier and re-dissolution theron. Thus, when used either alone or in combination with standard extraction and chromatographic techniques, the extractive methods of the invention allow for the isolation of cyclic peptides of the subject invention in high yield and high purity with fewer steps than are required by conventional methods.

### OBJECTIVES OF THE PRESENT INVENTION

The principal object of the present invention is to provide a method for purifying a peptide from a mixture containing atleast one related impurity.

Another object of the present invention is to provide a method of purification using the Reverse Phase High Performance Liquid Chromatography and Ion-Exchange chromatography.

Yet another object of the present invention is to provide a method for the purification of peptides which are cyclic or non-cyclic selected from a group comprising eptifibatide, exenatide, atosiban or nesiritide and related analogues or derivatives.

### STATEMENT OF THE INVENTION

Accordingly, the present invention is in relation to a method of purifying a peptide from a mixture containing at least one related impurity, said method comprising step of contacting the peptide mixture with Reverse Phase-High Performance Liquid Chromatographic matrix and/or ion-exchange chromatographic matrix to obtain purified peptide; a method of purifying a peptide using Reverse Phase-High Performance Liquid Chromatography from a mixture containing at least one related impurity, said method comprising steps of: packing the Reverse Phase-High Performance Liquid Chromatographic column with silica based polymer resin followed by equilibrating it with about 5% of polar solvent in an organic acid buffer; loading the peptide composition containing atleast one related impurity on the chromatographic column at a flow rate of about ≦100-400 cm/hr; washing the column with the same buffer solution as in Step (a); and eluting the purified product from the column by performing a linear gradient of 8-14% to obtain purified peptide product; a method of purifying peptide using ion-exchange chromatography from a mixture containing at least one related impurity, said method comprising steps of: equilibrating the cation-exchange column with an aqueous solution of a weak acid buffer; loading the Reverse Phase-High Performance Liquid Chromatographic column with purified peptide; and washing the column and eluting the peptide with the buffer as used in step (a) to obtain purified peptide product; and a method of purifying a peptide from a mixture containing at least one related impurity said method comprising steps of: packing Reverse Phase-High Performance Liquid Chromatographic column with a silica based polymer resin followed by equilibrating it with about 5% of polar solvent in an organic acid buffer; loading the peptide composition containing atleast one related impurity on the column at a flow rate of about ≦100-400 cm/hr followed by washing the column with the same buffer solution as in Step (a); eluting the purified product from the column by performing a linear gradient of 8-14%; loading the eluted product from Reverse Phase-High Performance Liquid Chromatographic column onto the cation-exchange column equilibrated with an aqueous solution of a weak acid buffer; and washing the column and eluting the peptide product in elution buffer to obtain the purified peptide product.

### BRIEF DESCRIPTION OF THE ACCOMPANYING DRAWINGS

FIG. 1. Chromatogram representing purity of eptifibatide

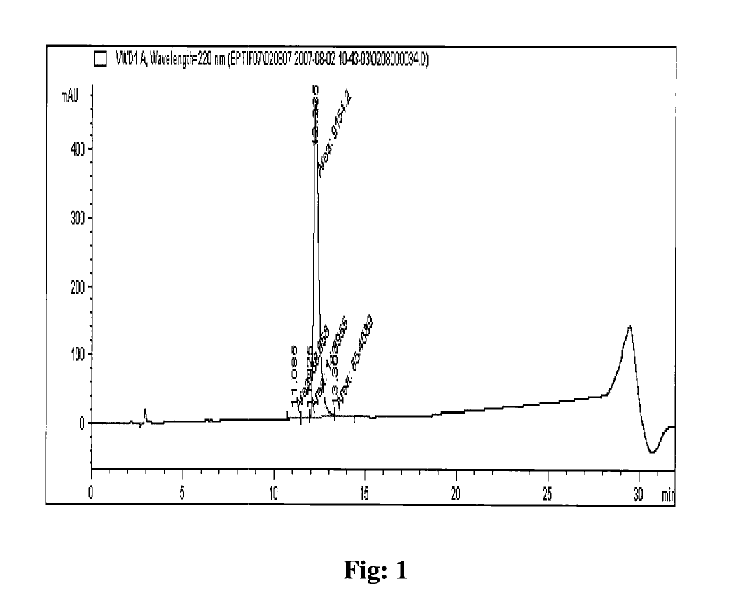

FIG. 2. Chromatogram representing purity of atosiban

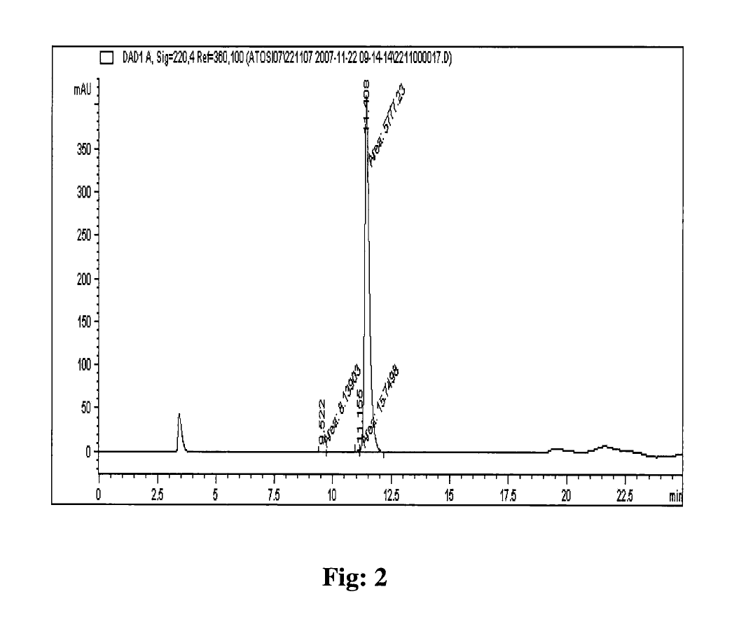

FIG. 3. Chromatogram representing purity of nesiritide.

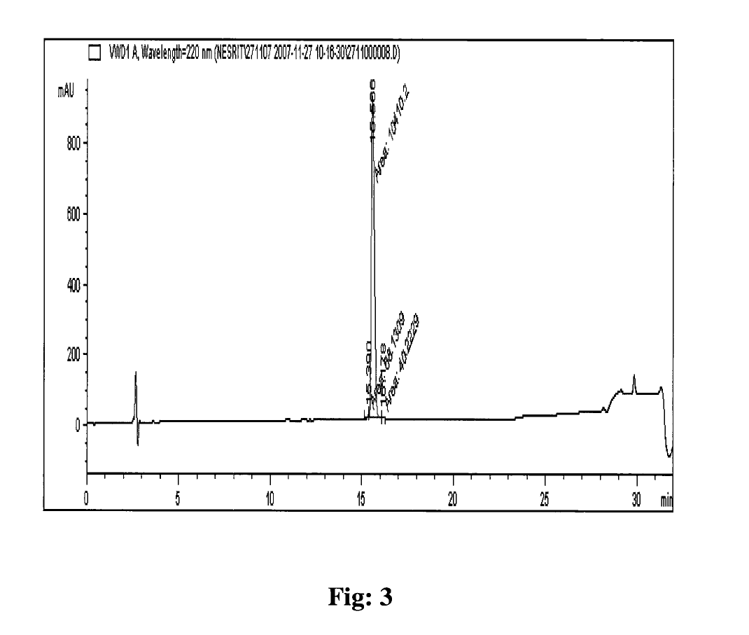

FIG. 4. Chromatogram representing purity of exenatide.

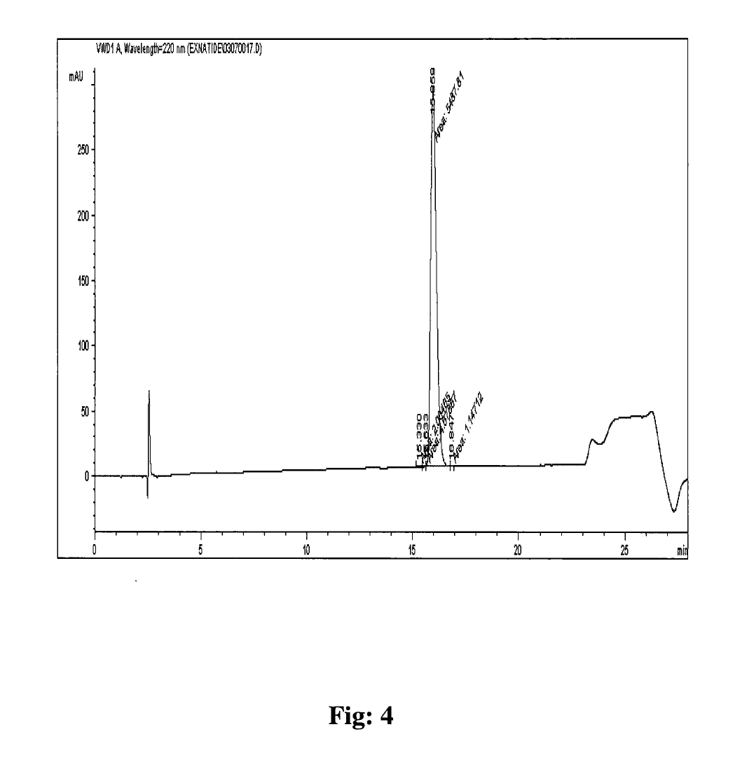

### DETAILED DESCRIPTION OF THE PRESENT INVENTION

The present invention is in relation to a method of purifying a peptide from a mixture containing at least one related impurity, said method comprising step of contacting the peptide mixture with Reverse Phase-High Performance Liquid Chromatographic matrix and/or ion-exchange chromatographic matrix to obtain purified peptide.

In another embodiment of the present invention said peptide is a cyclic or non-cyclic peptide selected from the group comprising eptifibatide, exenatide, atosiban or nesiritide and related analogs or derivatives.

Yet another embodiment of the present invention said peptide mixture is contacted in any sequence of order with said matrix composed of silica based polymeric resin.

Still another embodiment of the present invention the resin may be selected from a group comprising Sephadex, Sephadex LH20, Sephadex G-25, Sephadex G-10, Sepharose, Superdex, methylacrylate resin, carboxymethyl cellulose, sulphopropyl cellulose, carboxymethyl sephadex, sulphopropyl sephadex, sulphopropyl sepharose and carboxymethyl sepharose, preferably polystyrene or divinylbenzene.

Still another embodiment of the present invention the particle size and pore size of resin beads are ranging from 1 μm-50 μm and 100 Å-500 Å respectively.

Still another embodiment of the present invention said purification employs a gradient of about 2% to about 30% of a polar organic buffer solvent, in an aqueous phase containing an organic acid buffer.

Still another embodiment of the present invention wherein said polar buffer solvent is acetonitrile.

Still another embodiment of the present invention said organic acid buffer is selected from a group comprising citric acid, acetic acid, perchloric acid and formic acid.

In another embodiment of the present invention molarity of the buffer is ranging from 10 mM-50 mM.

In another embodiment of the present invention the purification is carried out at a pH ranging from 2-9.

In another embodiment of the present invention said method further comprising another optional step of size-exclusion chromatography.

In another embodiment of the present invention a purified peptide product obtained by a method of instant invention, wherein purity of the peptide product is ranging from 97%-100%.

In another embodiment of the present invention the purity of the product is atleast 96%.

The present invention is in relation to a method of purifying a peptide using Reverse Phase-High Performance Liquid Chromatography from a mixture containing at least one related impurity, said method comprising steps of:
a) packing the Reverse Phase-High Performance Liquid Chromatographic column with silica based polymer resin followed by equilibrating it with about 5% of polar solvent in an organic acid buffer;
b) loading the peptide composition containing atleast one related impurity on the chromatographic column at a flow rate of about ≦100-400 cm/hr;
c) washing the column with the same buffer solution as in Step (a); and
d) eluting the purified product from the column by performing a linear gradient of 8-14% to obtain purified peptide product.

The present invention is in relation to a method of purifying peptide using ion-exchange chromatography from a mixture containing at least one related impurity, said method comprising steps of:
a) equilibrating the cation-exchange column with an aqueous solution of a weak acid buffer;
b) loading the Reverse Phase-High Performance Liquid Chromatographic column with purified peptide; and
c) washing the column and eluting the peptide with the buffer as used in step (a) to obtain purified peptide product.

The present invention is in relation to a method of purifying a peptide from a mixture containing at least one related impurity said method comprising steps of:
a) packing Reverse Phase-High Performance Liquid Chromatographic column with a silica based polymer resin followed by equilibrating it with about 5% of polar solvent in an organic acid buffer;
b) loading the peptide composition containing atleast one related impurity on the column at a flow rate of about ≦100-400 cm/hr followed by washing the column with the same buffer solution as in Step (a);
c) eluting the purified product from the column by performing a linear gradient of 8-14%;
d) loading the eluted product from Reverse Phase-High Performance Liquid Chromatographic column onto the cation-exchange column equilibrated with an aqueous solution of a weak acid buffer; and
e) washing the column and eluting the peptide product in elution buffer to obtain the purified peptide product.

In another embodiment of the present invention the resin may be selected from a group comprising Sephadex, methylacrylate resin, carboxymethyl cellulose, carboxymethyl sephadex, sulphopropyl cellulose, and sulphopropyl sephadex, preferably polystyrene or divinylbenzene.

Yet another embodiment of the present invention the particle size and pore size of resin beads are ranging from 1 μm-50 μm and 100 Å-500 Å respectively.

Still another embodiment of the present invention said method further comprising another optional step of size-exclusion chromatography.

Still another embodiment of the present invention said peptide is a cyclic or non-cyclic peptide selected from the group comprising eptifibatide, exenatide, atosiban or nesiritide and related analogs or derivatives.

Still another embodiment of the present invention the polar buffer solvent is acetonitrile.

Still another embodiment of the present invention the organic acid buffer is selected from a group comprising citric acid, acetic acid and formic acid.

Still another embodiment of the present invention molarity of the buffer used is ranging from 10 mM-50 mM.

Still another embodiment of the present invention the purification is carried out at a pH ranging from 2-9.

Still another embodiment of the present invention A purified peptide product obtained by the methods of instant invention, wherein the purity of the peptide product is ranging from 97%-100%.

Still another embodiment of the present invention the peptide eptifibatide, exenatide, atosiban and nesiritide are associated with purity of atleast 96%.

The above and other objects of the present invention are provided in a specifically delineated process for forming and purifying cyclic or non-cyclic peptide compounds obtained after solid phase synthesis.

A method for the purification of peptides, the said process comprises chromatographic purification via reversed phase high performance liquid chromatography by employing a gradient of about 2% to about 20% polar buffer solvent, preferably acetonitrile in an aqueous phase containing an organic acid buffer at a pH between about 2 and about 5.

It is an object of the present invention to provide a chromatographic medium/solvent system wherein the process is based upon RP-HPLC and cation-exchange chromatography.

In a broad aspect, the present invention relates to a RP-HPLC chromatography process for purifying a peptide from a mixture comprising said peptide and related impurities, comprising the steps of: Separating said peptide and said related impurities of said mixture by elution though a RP-HPLC column packed with a polymer based resin equilibrated with about 5% of a polar solvent in an organic acid buffer, loading solution of the peptide on the column at a desired flow rate of ≦360 cm/hr,washing the column with a lower percentage (5%) of polar organic solvent in 50 mM organic acid, eluting the peptide product by performing a linear gradient of 8-14% with said combination of buffers.

Those skilled in the art will recognize that there are various variables which can be adjusted during the chromatographic procedures of the present invention. Such variables include loading and eluting conditions, such as ionic strength, buffer composition, pH, temperature, addition of a small amount of an organic solvent, etc. However, such variables are routinely adjusted in this field and those skilled in the art can readily establish optimum conditions.

Before explaining at least one embodiment of the invention in detail by way of exemplary drawings, experimentation, results, and procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting.

This invention relates to processes and procedures useful in purifying cyclic/non-cyclic peptides having biological activities similar to that of the natural peptides; and more particularly, to processes which utilize RP-HPLC, chromatography for separating the active peptide substance from other substances which do not have such activity and so may be regarded as impurities. The purified peptide product is prepared in the formulation solution using cation-exchange chromatography or a combination of cation-exchange chromatography and size exclusion chromatography.

One aspect of the present invention provides a method of purifying a peptide, polypeptide or protein prepared by solid phase synthesis said method comprising subjecting the peptide sample to a first step of purification through reverse-phase chromatography comprising an polymer based resin matrix under chromatographic conditions sufficient for obtaining said peptide of nearly 99% purity and further subjecting the eluted peptide to cation-exchange chromatography for concentration in a formulation solution to prepare the drug product.

The elution of peptide in formulation solution has advantages as compared to the conventional method. The conventional method comprises of freeze drying the purified peptide and redissolving the freeze dried powder in a solution prior to addition of excipients. The elution of peptide in formulation solution avoids the operation of freeze drier and re-dissolution theron.

The invention features, in another aspect, a method of purifying a protein, the method including the steps of subjecting a mixture that includes the compound to a reverse phase HPLC column and eluting a sample that includes the peptide with an organic solvent, contacting the sample with an ion exchange resin under conditions that allow the compound to be bound to the resin, and washing the organic solvent from the resin with an aqueous buffer solution.

The effective performance of the present invention requires the individuation of right combination of the chromatographic matrix to be used, the pH value and the ionic strength of the buffer for efficient purifications

Every attribute of the chromatographic procedure has a significant role to play in obtaining the desired protein product. The buffer system used for the purification is able to improve the separation of the impurities and molecule based on the hydrophobicity of the compounds. The buffer system at that specified pH elutes the impurities early during the elution gradient. This is achieved as the impurities being less hydrophobic than the molecule of interest at the specified condition.

The pH of the buffer system influences separation combined with hydrophobicity of compounds. The change in pH attributed during different steps of chromatography separation, effect the mobility of compounds in the column.

The beads used for the separation is polystyrene/divinylbenzene of 10 μm particle size and 300 Å pore size. The load sample has the ability to bind to beads as the pore size controls the surface area accessible for the molecules. The beads have high pH stability as it is polymer based and the particle size would provide better resolution.

### Formulation Solution Composition

### Eptifibatide

1. Injection: Each ml consist of 2 mg eptifibatide, 5.25 mg Citric acid and solution pH 5.25 adjusted with acetic acid, water q.s.
2. Infusion: Each ml consists of 0.75 mg eptifibatide, 5.25 mg Citric acid and solution pH 5.25 adjusted with acetic acid, water q.s.

### Atosiban

1. Injection: Each ml consists of 7.5 mg eptifibatide, 50 mg mannitol and solution pH 4.5 adjusted with HCl, water q.s.

### Definitions of Terms

In describing and claiming the present invention, the following terminology will be used in accordance with the definitions set out herein.

The term ‘polypeptide’, ‘protein’, ‘peptide’ refers to a polymer of amino acids and does not refer to a specific length of the product; thus, peptides, oligopeptides, and proteins are included within the definition of polypeptide. This term also does not refer to or exclude post-expression modifications of the polypeptide although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. In one embodiment, the molecule is a polypeptide or their related analogs or derivatives thereof. Preferably, the polypeptide is a cyclic peptide. According to another preferred embodiment, the polypeptide is a non-cyclic peptide. In still another preferred embodiment, the polypeptide is selected from the group comprising eptifibatide, exenatide, atosiban or nesiritide.

The term “purifying” a peptide from a composition comprising the peptide and one or more contaminants thereby increasing the degree of purity of the peptide in the composition by reducing the contents of at least one contaminant from the peptide composition.

An “impurity” is a material that is different from the desired polypeptide product or protein of interest. The impurity includes, but is not limited to diastereomers, hydrolysis products of labile amide bonds, deletion sequences formed predominantly in solid phase peptide synthesis, insertion peptides and by-products such as polymorphic forms that are formed during removal of protection groups in the final step of the synthesis.

The term “chromatography” refers to the process by which a solute of interest in a mixture is separated from other solutes in a mixture as a result of differences in rates at which the individual solutes of the mixture migrate through a stationary medium under the influence of a moving phase, or in bind and elute processes.

The term “High Performance liquid chromatography”, as used herein, refers to that chromatographic procedure in which the particles (stationary phase) used in the column packing are small (between 3 and 50 microns) and regular with little variation from the selected size. Such chromatography typically employs relatively high (around 500-3500 psi) inlet pressures.

The term “ion-exchange” and “ion-exchange chromatography” refers to the chromatographic process in which a solute of interest (such as a protein) in a mixture interacts with a charged compound linked (such as by covalent attachment) to a solid phase ion exchange material such that the solute of interest interacts non-specifically with the charged compound more or less than solute impurities or contaminants in the mixture. The contaminating solutes in the mixture elute from a column of the ion exchange material faster or slower than the solute of interest or are bound to or excluded from the resin relative to the solute of interest. “Ion-exchange chromatography” specifically includes cation exchange, anion exchange, and mixed mode chromatography. The Cation-exchange chromatography step may follow the RP-HPLC step, or vice versa. Preferably, the cation exchange chromatography step is followed by other chromatographic steps.

“Cation exchange chromatography” is a process in which positively charged ions bind to a negatively charged resin.

The “loading buffer” is that which is used to load the composition comprising the polypeptide molecule of interest and one or more impurities onto the ion exchange resin. The loading buffer has a conductivity and/or pH such that the polypeptide molecule of interest (and generally one or more impurities) is/are bound to the ion exchange resin or such that the protein of interest flows through the column while the impurities bind to the resin.

“Polar buffer solvent” may be any solvent that can dissolve ionic compounds or covalent compounds that ionize used as a buffer. Preferably in the context of the present invention the polar solvent used is acetonitrile.

A typical example of a method for the separation and purification of peptides of the invention prepared by solid phase synthesis comprises of the following steps:
i. Packing the RP-HPLC column with a polymer based resin equilibrated with about 5% of a polar solvent in an organic acid buffer.
ii. Loading the peptide composition containing atleast one related impurity on the column at a flow rate of about ≦100-400 cm/hr.
iii. Washing the column with the same buffer solution as in Step i.
iv. Eluting the purified product from the column performing a linear gradient of 8-14%.

The eluted purified solution of the peptide is further loaded on to a cation-exchange column to facilitate concentration of the product.

Concentration of said peptide combined with elution in formulation buffer through cation exchange chromatography comprises the following steps:
a. Equilibrating the cation-exchange column with an aqueous solution of a weak acid buffer.
b. Loading the RP-HPLC purified peptide onto the column

c. Washing the column and eluting the peptide product with the buffer as used in step a.

Buffer A is 1-5% acetonitrile, and Buffer B 10-50 mM organic acid buffer and the sample is loaded at a flow rate of about ≦100-400 cm/hr. The gradients used are subject to variation with respect the sample peptide to be purified.

The first step of the process herein involves purifying molecules from mixtures containing them by loading the mixtures on a reversed-phase liquid chromatography column. The column may be low-pressure or high-pressure (HPLC), the latter of which is packed with a medium having a particle diameter less than about 20 μm. Preferably, the column is packed with a medium having a particle diameter of about 5-40 μm, more preferably about 10-40 μm, and most preferably about 10-15 μm. In context of the present invention the particle size of the resin packed in the column is 10 μm. Hence, the column is preferably an HPLC column, especially for purification of peptides that require it. Preferably, the column has a pore size of about 100-4000 angstroms, more preferably about 100-500 angstroms. In context of the present invention the pore size of the resin packed in the column is 300 angstroms. The column length is preferably 10-50 cm, more preferably about 25-35 cm.

The pH of the elution buffer may be from about 2 to about 9, alternatively from about 3 to about 8, from about 4 to about 8, or from about 5 to about 8, although the pH or pH range for elution will be determined according to the desired protein of interest and the type of chromatography practiced. Appropriate pH ranges for a loading, wash, or elution buffer are readily determined by standard methods such that the protein of interest is recovered in an active form. Examples of elution buffers for this purpose include citrate or acetate buffers.

The medium of the column may be any suitable material, including polymeric-based resin media, silica-based media, or methacrylate media. Preferably, the medium is AMBERCHROM HPR10. Cation exchange resins contemplated for use in the practice of the present invention include sulphopropyl sepharose, hydrogel polymerized ceramic bead, carboxymethyl cellulose, hydrophilic spherical polymer beads, carboxymethyl sephadex, sulphopropyl cellulose, sulphopropyl sephadex, and the like. Presently preferred cation exchange resins include sulphopropyl sepharose and hydrogel polymerized ceramic bead, with sulphopropyl sepharose being the presently most preferred cation exchange resin for use in the practice of the present invention because of its ready availability and excellent performance. Size exclusion resins used are Sephadex LH-20, Sephadex G-25, Sephadex G-10.

The flow rate is generally about 20-400 cm/hour, or 4-40 column volumes (CV)/hour, depending on whether the chromatography is acidic or neutral. Preferably, the peptide is loaded on the column at a flow rate of ≦360 cm/hr.

The loading capacity of peptide on the column is generally 2-15 g/L for the RP-HPLC chromatographic separations based on the molecule and the impurities present. The loading capacity for ion-exchange columns is ≦70 g/L for the concentration step.

These and other non-limiting embodiments of the present invention are readily understood by one of ordinary skill in the art upon reading the disclosure and claims provided herein. It is understood that this invention is not limited to the particular methods and processes described, as such desired protein/peptide products and methods may, of course, vary. It is also to be understood that the terminology used herein is for the purpose of describing particular embodiments only, and is not intended to be limiting.

It will be appreciated that the use of the exemplified method of purification as described in the Examples to obtain high resolution separations, coupled with the components used for separation, makes it especially efficient for obtaining the desired peptide in a particularly simple, convenient and inexpensive manner.

The technology of the instant Application is further elaborated with the help of following examples. However, the examples should not be construed to limit the scope of the invention. The following Examples represent preferred embodiments of the present invention.

### Example 1

The eptifibatide TFA salt of 66% purity prepared by solid phase synthesis was used for purification on polymer based resin packed column. The eptifibatide TFA salt was dissolved initially in a mixture of 1:1, acetonitrile and 50 mM acetic acid to obtain a clear solution. The solution obtained was diluted further using 50 mM acetic acid to acetonitrile concentration of 5% and eptifibatide concentration of <2 g/L. The solution was filtered to load on the column.

The column packed with Amberchrom HPR10 (particle size 10 μm and pore size 300 Å) resin was equilibrated with lower percentage (5%) of acetonitrile in 50 mM acetic acid. The filtered solution of eptifibatide was loaded on the column at a flow rate of ≦360 cm/hr. The peptide loading on the column was performed to concentration of <10 g/L of resin. The column was washed after loading with lower percentage (5%) of acetonitrile in 50 mM acetic acid solution. The pure product was eluted from the column by performing a linear gradient of 8-14% of Acetonitrile (buffer B) for 25 CVs while 50 mM acetic acid is buffer A. The pressure drop across the column during the purification was 23-26 bars. The eptifibatide obtained from this process is of purity 98.7% with yield of 54%. The conditions for HPLC analysis of eptifibatide is given in the below table,

TABLE 1

| | | Gradient table | |
|---|---|---|---|
| Analytical method Conditions | | Time (min) | % B |
| Buffer A | 0.1% TFA + Water | 0 | 20 |
| BufferB | 0.1% TFA + Acetonitrile | 15 | 30 |
| Column | SS ExsilODS (80 A°, 5 μm), | 25 | 50 |
| Flow rate | 1 ml/min | 26 | 80 |
| Column temperature | 25° C. | 27 | 20 |
| Wavelength | 220 nm | 32 | 20 |

The purified solution of eptifibatide was loaded on a cation exchange column to facilitate the concentration of eptifibatide and perform elution into formulation buffer which would be the drug product concentrate. The eluate can be diluted for the required concentration and filled as drug product into vials.

The cation exchange column was equilibrated with 27 mM citric acid pH 2.70. The purified eptifibatide was loaded on a cation exchange column after diluting 1:1 with water to concentration of ≧65 g/L of matrix. The column was washed with 27 mM citric acid pH 2.70. The elution was performed using 27 mM citric acid pH 5.25. The eluate obtained was of concentration >9 g/L which was diluted to the required concentration to be filled in the vials as the drug product. The pressure drop across the column during the process is 0.5 to 0.7 bars at a flow rate of 180 cm/hr. The eptifibatide obtained from this process is of purity 98.6% with yield of 100%.

### Example 2

The eptifibatide TFA salt of 69.5% purity prepared by solid phase synthesis was used for purification on polymer based resin packed column. The eptifibatide TFA salt was dissolved initially in a mixture of 1:1, acetonitrile and 50 mM acetic acid to obtain a clear solution. The solution obtained was diluted further using 50 mM acetic acid to acetonitrile concentration of 5% and eptifibatide concentration of <2 g/L. The solution was filtered to load on the column. The pH of sodium acetate was adjusted with acetic acid to 3.0.

The column packed with Amberchrom HPR10 (particle size 10 μm and pore size 300 Å) resin was equilibrated with a lower percentage (5%) of acetonitrile in 10 mM sodium acetate pH 3.0. The filtered solution of eptifibatide was loaded on the column at a flow rate of ≦360 cm/hr. The peptide loading on the column was performed to concentration of <10 g/L of resin. The column was washed after loading with lower percentage (5%) of acetonitrile in 10 mM sodium acetate pH 3.0. The pure product was eluted from the column by performing a linear gradient of 9-12% of Acetonitrile (buffer B) for 25 CVs while 10 mM sodium acetate pH 3.0 is buffer A. The pressure drop across the column during the purification was 25-29 bars. The eptifibatide obtained from this process is of purity 98.6% with yield of 43.0%.

The purified solution of eptifibatide was loaded on a cation exchange column to facilitate the concentration of eptifibatide and perform elution into formulation buffer which would be the drug product concentrate. The eluate can be diluted for the required concentration and filled as drug product into vials.

The cation exchange column was equilibrated with 27 mM citric acid pH 2.70. The purified eptifibatide was loaded on a cation exchange column after diluting 1:1 with water to concentration of ≧65 g/L of matrix. The column was washed with 27 mM citric acid at pH 2.70. The elution was performed using 27 mM citric acid pH 5.25. The eluate obtained was of concentration >9 g/L which was diluted to the required concentration to be filled in the vials as drug product. The pressure drop across the column during the process was 0.5 to 0.7 bars at a flow rate of 180 cm/hr. The eptifibatide product obtained from this process is of purity 98.6% with yield of 100%.

### Example 3

The eptifibatide TFA salt of 69.5% purity prepared by solid phase synthesis was used for purification on polymer based resin packed column. The eptifibatide TFA salt was dissolved initially in a mixture of 1:1, acetonitrile and 50 mM acetic acid to obtain a clear solution. The solution obtained was diluted further using 50 mM acetic acid to acetonitrile concentration of 5% and eptifibatide concentration of <2 g/L. The solution was filtered to load on the column.

The column packed with Amberchrom HPR10 (particle size 10 μm and pore size 300 Å) resin was equilibrated with lower percentage (5%) of acetonitrile in 10 mM citric acid pH 2.5. The filtered solution of eptifibatide was loaded on the column at a flow rate of ≦360 cm/hr. The peptide loading on the column was performed to concentration of <10 g/L of resin. The column was washed after loading with lower percentage (5%) of acetonitrile in 10 mM citric acid pH 2.5. The pure product was eluted from the column by performing a linear gradient of 9-12% of Acetonitrile (buffer B) for 25 CVs while 10 mM citric acid pH 2.5 is buffer A. The pressure drop across the column during the purification was 22 to 28 bars. The eptifibatide obtained from this process is of purity 98.6% with yield of 57%.

The purified solution of eptifibatide was loaded on a cation exchange column to facilitate the concentration of eptifibatide and perform elution into formulation buffer which would be the drug product concentrate. The eluate can be diluted for the required concentration and filled as drug product into vials.

The cation exchange column was equilibrated with 27 mM citric acid pH 2.70. The purified eptifibatide was loaded on a cation exchange column after diluting 1:1 with water to concentration of ≧50 g/L of matrix. The column was washed with 27 mM citric acid pH 2.70. The elution was performed using 27 mM citric acid pH 5.25. The eluate obtained was of concentration >9 g/L which was diluted to the required concentration to be filled in the vials as drug product. The pressure drop across the column during the process was 0.5 to 0.7 bars at a flow rate of 180 cm/hr. The eptifibatide obtained from this process is of purity 98.6% with yield of 100%

### Example 4

The eptifibatide TFA salt of 66% purity prepared by solid phase synthesis was used for purification on polymer based resin packed column. The eptifibatide TFA salt was dissolved initially in a mixture of 1:1, acetonitrile and 50 mM acetic acid to obtain a clear solution. The solution obtained was diluted further using 50 mM acetic acid to acetonitrile concentration of 5% and eptifibatide concentration of <2 g/L. The solution was filtered to load on the column.

The column packed with Amberchrom HPR10 (particle size 10 μm and pore size 300 Å) resin was equilibrated with lower percentage (5%) of acetonitrile in 0.05% Perchloric acid pH 1.70. The filtered solution of eptifibatide was loaded on the column at a flow rate of ≦360 cm/hr. The peptide loading on the column was performed to concentration of <10 g/L of resin. The column was washed after loading with lower percentage (5%) of acetonitrile in 0.05% Perchloric acid pH 1.70. The pure product was eluted from the column by performing a linear gradient of 8-12% of Acetonitrile (buffer B) for 25 CVs while 0.05% Perchloric acid pH 1.70 is buffer A. The pressure drop across the column during the purification was 28-32 bars. The eptifibatide obtained from this process is of purity 96.6% with yield of 40%.

The purified solution of eptifibatide was loaded on a cation exchange column to facilitate the concentration of eptifibatide and perform elution into formulation buffer which would be the drug product concentrate. The eluate can be diluted for the required concentration and filled as drug product into vials.

The cation exchange column was equilibrated with 27 mM citric acid pH 2.70. The purified eptifibatide was loaded on a cation exchange column after diluting 1:1 with water to concentration of ≧50 g/L of matrix. The column was washed with 27 mM citric acid pH 2.70. The elution was performed using 27 mM citric acid pH 5.25. The eluate obtained was of concentration >9 g/L which was diluted to the required concentration to be filled in the vials as drug product. The pressure drop across the column during the process was 0.5 to 0.7 bars at a flow rate of 180 cm/hr. The eptifibatide obtained from this process is of purity 98.6% with yield of 100%.

### Example 5

The eptifibatide TFA salt of 69.5% purity prepared by solid phase synthesis was used for purification on polymer based resin packed column. The eptifibatide TFA salt was dissolved initially in a mixture of 1:1, acetonitrile and 50 mM acetic acid to obtain a clear solution. The solution obtained was diluted further using 50 mM acetic acid to acetonitrile concentration of 5% and eptifibatide concentration of <2 g/L. The solution was filtered to load on the column.

The column packed with Amberchrom HPR10 (particle size 10 μm and pore size 300 Å) resin was equilibrated with lower percentage (5%) of acetonitrile in 10 mM sodium formate pH 3.0. The filtered solution of eptifibatide was loaded on the column at a flow rate of ≦360 cm/hr. The peptide loading on the column was performed to concentration of <10 g/L of resin. The column was washed after loading with lower percentage (5%) of acetonitrile in 10 mM sodium formate pH 3.0. The pure product was eluted from the column by performing a linear gradient of 5-17% of Acetonitrile (buffer B) for 25 CVs while 10 mM sodium formate pH 3.0 is buffer A. The pressure drop across the column during the purification was 30-33 bars. The eptifibatide obtained from this process is of purity 96.8% with yield of 54.0%.

The purified solution of eptifibatide was loaded on a cation exchange column to facilitate the concentration of eptifibatide and perform elution into formulation buffer which would be the drug product concentrate. The eluate can be diluted for the required concentration and filled as drug product into vials.

The cation exchange column was equilibrated with 27 mM citric acid pH 2.70. The purified eptifibatide was loaded on a cation exchange column after diluting 1:1 with water to concentration of ≧50 g/L of matrix. The column was washed with 27 mM citric acid pH 2.70. The elution was performed using 27 mM citric acid pH 5.25. The eluate obtained was of concentration >9 g/L which was diluted to the required concentration to be filled in the vials as drug product. The pressure drop across the column during the process was 0.5 to 0.7 bars at a flow rate of 180 cm/hr. The eptifibatide obtained from this process is of purity 98.6% with yield of 100%.

### Example 6

The eptifibatide TFA salt of 69.5% purity prepared by solid phase synthesis was used for purification on polymer based resin packed column. The eptifibatide TFA salt was dissolved initially in a mixture of 1:1, acetonitrile and 50 mM acetic acid to obtain a clear solution. The solution obtained was diluted further using 50 mM acetic acid to acetonitrile concentration of 5% and eptifibatide concentration of <2 g/L. The solution was filtered to load on the column.

The column packed with Amberchrom HPR10 (particle size 10 μm and pore size 300 Å) resin was equilibrated with lower percentage (5%) of acetonitrile in 10 mM Boric acid pH 4.0. The filtered solution of eptifibatide was loaded on the column at a flow rate of ≦360 cm/hr. The peptide loading on the column was performed to concentration of <10 g/L of resin. The column was washed after loading with lower percentage (5%) of acetonitrile in 10 mM Boric acid pH 4.0. The pure product was eluted from the column by performing a linear gradient of 5-17% of Acetonitrile (buffer B) for 25 CVs while 10 mM Boric acid pH 4.0 is buffer A. The pressure drop across the column during the purification was 30-33 bars. The eptifibatide obtained from this process is of purity 98.0% with yield of 51.0%.

The purified solution of eptifibatide was loaded on a cation exchange column to facilitate the concentration of eptifibatide and perform elution into formulation buffer which would be the drug product concentrate. The eluate can be diluted for the required concentration and filled as drug product into vials.

The cation exchange column was equilibrated with 27 mM citric acid pH 2.70. The purified eptifibatide was loaded on a cation exchange column after diluting 1:1 with water to concentration of ≧50 g/L of matrix. The column was washed with 27 mM citric acid pH 2.70. The elution was performed using 27 mM citric acid pH 5.25. The eluate obtained was of concentration >9 g/L which was diluted to the required concentration to be filled in the vials as drug product. The pressure drop across the column during the process was 0.5 to 0.7 bars at a flow rate of 180 cm/hr. The eptifibatide obtained from this process is of purity 98.6% with yield of 100%

### Example 7

### Atosiban

The atosiban crude salt of 73.5% purity prepared by solid phase synthesis was used for purification on polymer based resin packed column. The atosiban crude salt was dissolved initially in a mixture of 1:1, acetonitrile and 50 mM acetic acid to obtain a clear solution. The solution obtained was diluted further using 50 mM acetic acid to acetonitrile concentration of 5% and atosiban concentration of <2 g/L. The solution was filtered to load on the column.

The column packed with Amberchrom HPR10 (particle size 10 μm and pore size 300 Å) resin was equilibrated with lower percentage (5%) of acetonitrile in 50 mM acetic acid. The filtered solution of atosiban was loaded on the column at a flow rate of ≦360 cm/hr. The peptide loading on the column was performed to concentration of <10 g/L of resin. The column was washed after loading with lower percentage (9%) of acetonitrile in 50 mM acetic acid. The pure product was eluted from the column by performing a linear gradient of 9-12% of Acetonitrile (buffer B) for 25 CVs while 50 mM acetic acid was buffer A. The pressure drop across the column during the purification is 26-32 bars. The eptifibatide obtained from this process is of purity 98.6% with yield of 57%. The conditions for HPLC analysis of atosiban is given in the below table,

TABLE 2

| | | Gradient table | |
|---|---|---|---|
| Analytical method Conditions | | Time (min) | % B |
| Buffer A | 0.18% Et3N + Water adjusted to pH | 0 | 24 |
| | 2.3 with OPA | | |
| Buffer B | Acetonitrile | 15.0 | 38 |
| Column | Supelco (180 A°, 5 μm) | 15.1 | 50 |
| Flow rate | 1 ml/min | 19.0 | 50 |
| Column | 25° C. | 19.1 | 24 |
| Temperature | | | |
| Wavelength | 220 nm | 25.0 | 24 |

### Example 8

The atosiban crude salt of 73.5% purity prepared by solid phase synthesis was used for purification on polymer based resin packed column. The atosiban crude powder was dissolved in a mixture of 5% acetonitrile and 50 mM acetic acid to obtain a clear solution and product concentration <2 g/L. The solution was filtered to load on the column.

The column packed with Amberchrom HPR10 (particle size 10 μm and pore size 300 Å) resin was equilibrated with lower percentage (5%) of acetonitrile in 50 mM acetic acid. The filtered solution of atosiban was loaded on the column at a flow rate of ≦360 cm/hr. The peptide loading on the column is performed to concentration of ≦10 g/L of resin. The column was washed after loading with lower percentage (5% and 9%) of acetonitrile in 50 mM acetic acid. The pure product was eluted from the column by performing a linear gradient of 9-13% of Acetonitrile (buffer B) for 25 CVs while 50 mM acetic acid is buffer A. The pressure drop across the column during the purification is 26-32 bars. The atosiban obtained from this process is of purity 99.6% with yield of 71.0%.

The purified solution of atosiban was loaded on a cation exchange column to facilitate the concentration. The cation exchange (CIEX) column was equilibrated with 5 mM acetic acid pH 3.3. The purified atosiban was loaded on a cation exchange column after diluting 1:1 with water. The loading on the column is ≦50 g/L of resin. The column was washed with 5 mM acetic acid pH 3.3. The elution was performed using 500 mM ammonium acetate pH 7.8. The eluate obtained was of concentration >15 g/L. The pressure drop across the column during the process was 0.2 to 0.3 bar at a flow rate of 60 cm/hr. The atosiban obtained from this process was of purity 99.6% with yield of 80%.

The elution pool obtained from cation exchange column was injected into the size exclusion column to perform buffer exchange into acetic acid which would be the drug product concentrate. The eluate can be diluted for required concentration with formulation components and filled as drug product into vials.

The size exclusion column was equilibrated with low concentration of acetic acid (2-5 mM). The sample (CIEX elution pool) volume injected into the column was 30% of column volume. The elution from the column is collected such that the concentration of atosiban is >15 g/L. The process is run at a flow rate of 15 cm/hr with a pressure drop of <3 bar across the column. The eluate obtained from the column was the concentrate which was diluted to the required concentration with addition of formulation components to be filled in the vial.

### Example 9

The atosiban crude salt of 84.1% purity prepared by solid phase synthesis was used for purification on polymer based resin packed column. The atosiban crude powder was dissolved in a mixture of 5% acetonitrile and 50 mM acetic acid to obtain a clear solution and product concentration <2 g/L. The solution was filtered to load on the column.

The column packed with Amberchrom HPR10 (particle size 10 μm and pore size 300 Å) resin was equilibrated with lower percentage (5%) of acetonitrile in 10 mM sodium acetate pH 3.0. The filtered solution of atosiban was loaded on the column at a flow rate of ≦360 cm/hr. The peptide loading on the column was performed to concentration of ≦10 g/L of resin. The column was washed after loading with lower percentage (5%) of acetonitrile in 10 mM sodium acetate pH 3.0. The pure product was eluted from the column by performing a linear gradient of 13-20% of Acetonitrile (buffer B) for 20 CVs while 10 mM sodium acetate pH 3.0 was buffer A. The pressure drop across the column during the purification was 19-22 bars. The atosiban obtained from this process is of purity 99.6% with yield of 94.0%.

The purified solution of atosiban was loaded on a cation exchange column to facilitate the concentration. The cation exchange (CIEX) column was equilibrated with 5 mM acetic acid pH 3.3. The purified atosiban was loaded on a cation exchange column after diluting 1:1 with water. The loading on the column was ≦50 g/L of resin. The column was washed with 5 mM acetic acid pH 3.3. The elution was performed using 500 mM ammonium acetate pH 7.8. The eluate obtained was of concentration >15 g/L. The pressure drop across the column during the process was 0.2 to 0.3 bar at a flow rate of 60 cm/hr. The atosiban obtained from this process is of purity 99.6% with yield of 80%.

The elution pool obtained from cation exchange column is injected into the size exclusion column to perform buffer exchange into acetic acid which would be the drug product concentrate. The eluate can be diluted for required concentration with formulation components and filled as drug product into vials.

The size exclusion column is equilibrated with low concentration of acetic acid (2-5 mM). The sample (CIEX elution pool) volume injected into the column is 30% of column volume. The elution from the column was collected such that the concentration of atosiban was >15 g/L. The process was run at a flow rate of 15 cm/hr with a pressure drop of <3 bar across the column. The eluate obtained from the column was the concentrate which was diluted to the required concentration with addition of formulation components to be filled in the vial.

### Example 10

The atosiban crude salt of 81.5% purity prepared by solid phase synthesis was used for purification on polymer based resin packed column. The atosiban crude powder was dissolved in a mixture of 5% acetonitrile and 50 mM acetic acid to obtain a clear solution and product concentration <2 g/L. The solution was filtered to load on the column.

The column packed with Amberchrom HPR10 (particle size 10 μm and pore size 300 Å) resin was equilibrated with lower percentage (5%) of acetonitrile in 10 mM citric acid pH 3.0. The filtered solution of atosiban was loaded on the column at a flow rate of ≦360 cm/hr. The peptide loading on the column was performed to concentration of ≦10 g/L of resin. The column was washed after loading with lower percentage (5%) of acetonitrile in 10 mM citric acid pH 3.0. The pure product was eluted from the column by performing a linear gradient of 12-20% of Acetonitrile (buffer B) for 25 CVs while 10 mM citric acid pH 3.0 was buffer A. The pressure drop across the column during the purification was 22-23 bars. The atosiban obtained from this process was of purity 99.8% with yield of 73.0%.

The purified solution of atosiban was loaded on a cation exchange column to facilitate the concentration. The cation exchange (CIEX) column was equilibrated with 5 mM acetic acid pH 3.3. The purified atosiban was loaded on a cation exchange column after diluting 1:1 with water. The loading on the column was ≦50 g/L of resin. The column was washed with 5 mM acetic acid pH 3.3. The elution was performed using 500 mM ammonium acetate pH 7.8. The eluate obtained was of concentration >15 g/L. The pressure drop across the column during the process was 0.2 to 0.3 bars at a flow rate of 60 cm/hr. The atosiban obtained from this process is of purity 99.6% with yield of 80%.

The elution pool obtained from cation exchange column was injected into the size exclusion column to perform buffer exchange into acetic acid which would be the drug product concentrate. The eluate can be diluted for required concentration with formulation components and filled as drug product into vials.

The size exclusion column was equilibrated with low concentration of acetic acid (2-5 mM). The sample (CIEX elution pool) volume injected into the column was 30% of column volume. The elution from the column was collected such that the concentration of atosiban was >15 g/L. The process was run at a flow rate of 15 cm/hr with a pressure drop of <3 bar across the column. The eluate obtained from the column was the concentrate which was diluted to the required concentration with addition of formulation components to be filled in the vial.

### Example 11

The atosiban crude salt of 80.2% purity prepared by solid phase synthesis was used for purification on polymer based resin packed column. The atosiban crude powder was dissolved in a mixture of 5% acetonitrile and 50 mM acetic acid to obtain a clear solution and product concentration <2 g/L. The solution was filtered to load on the column.

The column packed with Amberchrom HPR10 (particle size 10 μm and pore size 300 Å) resin was equilibrated with lower percentage (5%) of acetonitrile in 0.05% Perchloric acid pH 1.70. The filtered solution of atosiban was loaded on the column at a flow rate of ≦360 cm/hr. The peptide loading on the column was performed to concentration of ≦10 g/L of resin. The column was washed after loading with lower percentage (5%) of acetonitrile in 0.05% Perchloric acid pH 1.70. The pure product was eluted from the column by performing a linear gradient of 12-20% of Acetonitrile (buffer B) for 25 CVs while 0.05% Perchloric acid pH 1.70 is buffer A. The pressure drop across the column during the purification was 23-24 bars. The atosiban obtained from this process is of purity 99.6% with yield of 94.0%.

The purified solution of atosiban was loaded on a cation exchange column to facilitate the concentration. The cation exchange (CIEX) column was equilibrated with 5 mM acetic acid pH 3.3. The purified atosiban was loaded on a cation exchange column after diluting 1:1 with water. The loading on the column was ≦50 g/L of resin. The column was washed with 5 mM acetic acid pH 3.3. The elution was performed using 500 mM ammonium acetate pH 7.8. The eluate obtained was of concentration >15 g/L. The pressure drop across the column during the process was 0.2 to 0.3 bars at a flow rate of 60 cm/hr. The atosiban obtained from this process was of purity 99.6% with yield of 80%.

The elution pool obtained from cation exchange column was injected into the size exclusion column to perform buffer exchange into acetic acid which would be the drug product concentrate. The eluate can be diluted for required concentration with formulation components and filled as drug product into vials.

The size exclusion column was equilibrated with low concentration of acetic acid (2-5 mM). The sample (CIEX elution pool) volume injected into the column was 30% of column volume. The elution from the column was collected such that the concentration of atosiban was >15 g/L. The process was run at a flow rate of 15 cm/hr with a pressure drop of <3 bar across the column. The eluate obtained from the column was the concentrate which was diluted to the required concentration with addition of formulation components to be filled in the vial.

### Example 12

The atosiban crude salt of 73.5% purity prepared by solid phase synthesis was used for purification on polymer based resin packed column. The atosiban crude powder was dissolved in a mixture of 5% acetonitrile and 50 mM acetic acid to obtain a clear solution and product concentration <2 g/L. The solution was filtered to load on the column.

The column packed with Amberchrom HPR10 (particle size 10 μm and pore size 300 Å) resin was equilibrated with lower percentage (5%) of acetonitrile in 0.05% Perchloric acid pH 3.0. The filtered solution of atosiban was loaded on the column at a flow rate of ≦360 cm/hr. The peptide loading on the column was performed to concentration of ≦10 g/L of resin. The column was washed after loading with lower percentage (5%) of acetonitrile in 0.05% Perchloric acid pH 3.0. The pure product was eluted from the column by performing a linear gradient of 12-20% of Acetonitrile (buffer B) for 25 CVs while 0.05% Perchloric acid pH 3.0 is buffer A. The pressure drop across the column during the purification was 26-32 bars. The atosiban obtained from this process is of purity 99.4% with yield of 71.0%.

The purified solution of atosiban was loaded on a cation exchange column to facilitate the concentration. The cation exchange (CIEX) column was equilibrated with 5 mM acetic acid pH 3.3. The purified atosiban was loaded on a cation exchange column after diluting 1:1 with water. The loading on the column was ≦50 g/L of resin. The column was washed with 5 mM acetic acid pH 3.3. The elution was performed using 500 mM ammonium acetate pH 7.8. The eluate obtained was of concentration >15 g/L. The pressure drop across the column during the process was 0.2 to 0.3 bars at a flow rate of 60 cm/hr. The atosiban obtained from this process was of purity 99.6% with yield of 80%.

The elution pool obtained from cation exchange column was injected into the size exclusion column to perform buffer exchange into acetic acid which would be the drug product concentrate. The eluate can be diluted for required concentration with formulation components and filled as drug product into vials.

The size exclusion column was equilibrated with low concentration of acetic acid (2-5 mM). The sample (CIEX elution pool) volume injected into the column was 30% of column volume. The elution from the column was collected such that the concentration of atosiban was >15 g/L. The process was run at a flow rate of 15 cm/hr with a pressure drop of <3 bar across the column. The eluate obtained from the column was the concentrate which was diluted to the required concentration with addition of formulation components to be filled in the vial.

### Example 13

### Nesiritide

The nesiritide crude salt of 59.0% purity prepared by solid phase synthesis was used for purification on polymer based resin packed column. The nesiritide crude powder was dissolved in a mixture of 10% acetonitrile and 50 mM acetic acid to obtain a clear solution and product concentration <2 g/L. The solution is filtered to load on the column.

The column packed with Amberchrom HPR10 (particle size 10 μm and pore size 300 Å) resin was equilibrated with lower percentage (5%) of acetonitrile in 10 mM sodium acetate pH 3.0. The filtered solution of nesiritide was loaded on the column at a flow rate of ≦360 cm/hr. The peptide loading on the column was performed to concentration of ≦10 g/L of resin. The column was washed after loading with lower percentage (5%) of acetonitrile in 10 mM sodium acetate pH 3.0. The pure product was eluted from the column by performing a linear gradient of 5-15% of Acetonitrile (buffer B) for 25 CVs while 10 mM sodium acetate pH 3.0 is buffer A. The pressure drop across the column during the purification was 28-33 bars. The nesiritide obtained from this process was of purity 92.5% with yield of 50.0%. The conditions for HPLC analysis of nesiritide is given in the below table,

TABLE 3

| | | Gradient table | |
|---|---|---|---|
| Analytical method conditions | | Time (min) | % B |
| Buffer A | 0.1% TFA + Water | 0 | 10 |
| Buffer B | 0.1% TFA + Acetonitrile | 20.0 | 25 |
| Column | Symmetry C18 (300 Å, 5 m) | 25.0 | 50 |
| Flow rate | 1.2 ml/min | 25.1 | 80 |
| Column | 60° C. | 28.0 | 80 |
| Temperature | | | |
| Wavelength | 220 nm | 28.1 | 10 |
| | | 32.0 | 10 |

### Example 14

The nesiritide crude salt of 59.0% purity prepared by solid phase synthesis was used for purification on polymer based resin packed column. The nesiritide crude powder was dissolved in a mixture of 10% acetonitrile and 50 mM acetic acid to obtain a clear solution and product concentration <2 g/L. The solution was filtered to load on the column.

The column packed with Amberchrom HPR10 (particle size 10 μm and pore size 300 Å) resin was equilibrated with lower percentage (5%) of acetonitrile in 10 mM citric acid pH 3.0. The filtered solution of nesiritide was loaded on the column at a flow rate of ≦360 cm/hr. The peptide loading on the column was performed to concentration of ≦10 g/L of resin. The column was washed after loading with lower percentage (5%) of acetonitrile in 10 mM citric acid pH 3.0. The pure product was eluted from the column by performing a linear gradient of 5-15% of Acetonitrile (buffer B) for 25 CVs while 10 mM citric acid pH 3.0 was buffer A. The pressure drop across the column during the purification was 28-33 bars. The nesiritide obtained from this process is of purity 97.2% with yield of 33.0%.

### Example 15

The nesiritide crude salt of 59.0% purity prepared by solid phase synthesis was used for purification on polymer based resin packed column. The nesiritide crude powder was dissolved in a mixture of 10% acetonitrile and 50 mM acetic acid to obtain a clear solution and product concentration <2 g/L. The solution was filtered to load on the column.

The column packed with Amberchrom HPR10 (particle size 10 μm and pore size 300 Å) resin was equilibrated with lower percentage (5%) of acetonitrile in 10 mM sodium formate pH 3.0. The filtered solution of nesiritide was loaded on the column at a flow rate of ≦360 cm/hr. The peptide loading on the column was performed to concentration of ≦10 g/L of resin. The column was washed after loading with lower percentage (5%) of acetonitrile in 10 mM sodium formate pH 3.0. The pure product was eluted from the column by performing a linear gradient of 12-20% of Acetonitrile (buffer B) for 25 CVs while 10 mM sodium formate pH 3.0 is buffer A. The pressure drop across the column during the purification was 24-28 bars. The nesiritide obtained from this process is of purity 98.7% with yield of 18.0%.

### Example 16

### Exenatide

The exenatide TFA salt of 56% purity prepared by solid phase synthesis was used for purification on polymer based resin packed column. The exenatide TFA salt was dissolved initially in a mixture of 1:1, acetonitrile and 50 mM acetic acid to obtain a clear solution. The solution obtained was diluted further using 50 mM acetic acid to acetonitrile concentration of 5% and exenatide concentration of <2 g/L. The solution was filtered to load on the column.

The column packed with Amberchrom HPR10 (particle size 10 μm and pore size 300 Å) resin was equilibrated with lower percentage (5%) of acetonitrile in 10 mM citric acid pH 5.0. The filtered solution of exenatide was loaded on the column at a flow rate of ≦360 cm/hr. The peptide loading on the column was performed to concentration of <5 g/L of resin. The column was washed after loading with lower percentage (5%) of acetonitrile in 10 mM citric acid pH 5.0. The pure product was eluted from the column by performing a linear gradient of 30-33% of Acetonitrile (buffer B) for 20 CVs while 10 mM citric acid pH 5.0 was buffer A. The pressure drop across the column during the purification was 15-25 bar. The exenatide obtained from this process was of purity 92.2% with yield of 49%. The conditions for HPLC analysis of exenatide is given in the below table,

TABLE 4

| | | Gradient table | |
|---|---|---|---|
| Analytical method | | Time (min) | % B |
| Buffer A | 0.1% TFA + Water | 0 | 25 |
| Buffer B | 0.1% TFA + Acetonitrile | 10 | 35 |
| Column | Symmetry C18 (300 A, 5 m) | 20 | 40 |
| Flow rate | 1.2 ml/min | 21 | 70 |
| Column | 40° C. | 23 | 70 |
| Temperature | | | |
| Wavelength | 220 nm | 24 | 25 |
| | | 28 | 25 |

The elution pool obtained from the above process was diluted three times with water to facilitate binding on the column. The exenatide elution pool from example 1 of purity 92.0% is diluted three times with water to facilitate binding on column.

The column packed with Amberchrom HPR10 (particle size 10 μm and pore size 300 Å) resin from Rohm and Haas was equilibrated with 10% percentage of acetonitrile in 50 mM acetic acid. The sample prepared of 92.0% pure exenatide was loaded on the column at a flow rate of ≦360 cm/hr. The peptide loading on the column was performed to concentration of <5 g/L of resin. The column was washed after loading with 20% of acetonitrile in 50 mM acetic acid.

The pure product was eluted from the column by performing a linear gradient of 25-28% of Acetonitrile (buffer B) for 20 CVs while 50 mM acetic acid was buffer A. The pressure drop across the column during the purification is 20-25 bars. The exenatide obtained from this process was of purity 99.6% with yield of 50%.

### Example 17

The exenatide TFA salt of 46.2% purity prepared by solid phase synthesis was used for purification on polymer based resin packed column. The exenatide TFA salt was dissolved initially in a mixture of 1:1, acetonitrile and 50 mM acetic acid to obtain a clear solution. The solution obtained was diluted further using 50 mM acetic acid to acetonitrile concentration of 5% and exenatide concentration of <2 g/L. The solution was filtered to load on the column.

The column packed with Amberchrom HPR10 (particle size 10 μm and pore size 300 Å) resin was equilibrated with lower percentage (10%) of acetonitrile in 10 mM Sodium acetate pH 4.0. The filtered solution of exenatide was loaded on the column at a flow rate of ≦360 cm/hr. The peptide loading on the column was performed to concentration of <10 g/L of resin. The column was washed after loading with lower percentage (10%) of acetonitrile in 10 mM

Sodium acetate pH 4.0. The pure product was eluted from the column by performing a linear gradient of 28-33% of Acetonitrile (buffer B) for 20 CVs while 10 mM Sodium acetate pH 4.0 was buffer A. The pressure drop across the column during the purification was 18-20 bars. The exenatide obtained from this process was of purity 94.2% with yield of 57%.

The exenatide elution pool from above example, of purity 94.2% was diluted three times with water to facilitate binding on column.

The column packed with Amberchrom HPR10 (particle size 10 μm and pore size 300 Å) resin from Rohm and Haas was equilibrated with 10% percentage of acetonitrile in 50 mM acetic acid. The sample prepared of 94.2% pure exenatide was loaded on the column at a flow rate of ≦360 cm/hr. The peptide loading on the column was performed to concentration of <10 g/L of resin. The column was washed after loading with 20% of acetonitrile in 50 mM acetic acid. The pure product was eluted from the column by performing a linear gradient of 25-28% of Acetonitrile (buffer B) for 20 CVs while 50 mM acetic acid was buffer A. The pressure drop across the column during the purification was 20-25 bars. The exenatide obtained from this process was of purity 99.6% with yield of 50%.

### Example 18

The exenatide TFA salt of 46.2% purity prepared by solid phase synthesis was used for purification on polymer based resin packed column. The exenatide TFA salt was dissolved initially in a mixture of 1:1, acetonitrile and 50 mM acetic acid to obtain a clear solution. The solution obtained was diluted further using 50 mM acetic acid to acetonitrile concentration of 5% and exenatide concentration of <2 g/L. The solution was filtered to load on the column.

The column packed with Amberchrom HPR10 (particle size 10 μm and pore size 300 Å) resin was equilibrated with lower percentage (10%) of acetonitrile in 10 mM Sodium formate pH 4.0. The filtered solution of exenatide was loaded on the column at a flow rate of ≦360 cm/hr. The peptide loading on the column was performed to concentration of <10 g/L of resin. The column was washed after loading with lower percentage (10%) of acetonitrile in 10 mM Sodium formate pH 4.0. The pure product was eluted from the column by performing a linear gradient of 27-35% of Acetonitrile (buffer B) for 20 CVs while 10 mM Sodium formate pH 4.0 was buffer A. The pressure drop across the column during the purification was 24-29 bars. The exenatide obtained from this process is of purity 94.9% with yield of 52%.

The exenatide elution pool from above example, of purity 94.9% was diluted three times with water to facilitate binding on column. The column packed with Amberchrom HPR10 (particle size 10 μm and pore size 300 Å) resin from Rohm and Haas was equilibrated with 10% percentage of acetonitrile in 50 mM acetic acid. The sample prepared of 94.2% pure exenatide was loaded on the column at a flow rate of ≦360 cm/hr. The peptide loading on the column is performed to concentration of <10 g/L of resin. The column was washed after loading with 20% of acetonitrile in 50 mM acetic acid. The pure product was eluted from the column by performing a linear gradient of 25-28% of Acetonitrile (buffer B) for 20 CVs while 50 mM acetic acid was buffer A. The pressure drop across the column during the purification is 20-25 bars. The exenatide obtained from this process is of purity 99.6% with yield of 50%.

We claim:

1. A method of purifying a cyclic or non-cyclic peptide and related analogs or derivatives from a mixture containing at least one related impurity comprising contacting the said peptide mixture in any sequence of order, with a RP-HPLC chromatographic matrix and/or ion-exchange chromatographic matrix said separation matrix composed of silica based polymeric resin.

2. The method according to claim 1, wherein said purification is carried out employing a gradient of about 2% to about 30% of a polar organic buffer solvent, in an aqueous phase containing an organic acid buffer.

3. The method according to claim 1, wherein the resin may be selected from the group comprising Sephadex, Sephadex LH20, Sephadex G-25, Sephadex G-10, Sepharose, Superdex, methylacrylate resin, carboxymethyl cellulose, sulphopropyl cellulose, carboxymethyl sephadex, sulphopropyl sephadex, sulphopropyl sepharose, carboxymethyl sepharose

4. The method according to claim 1, wherein the resin is polystyrene or divinylbenzene.

5. The method according to claim 1, wherein the particle size and pore size of resin beads are ranging from 1 μm-50 μm and 100 Å-500 Å respectively.

6. The method according to claim 1, wherein the cyclic or non-cyclic peptide is selected from the group comprising eptifibatide, exenatide, atosiban or nesiritide.

7. The method according to claim 2, wherein the polar buffer solvent is acetonitrile.

8. The method according to claim 2, wherein the organic acid buffer is selected from the group comprising citric acid, acetic acid, perchloric acid and formic acid.

9. The method according to claim 2, wherein the molarity of the buffer used is ranging from 10 mM-50 mM.

10. The method according to any of the preceding claims wherein the purification is carried out at a pH ranging from 2-9.

11. The method according to claim 1, wherein said method further comprising another optional step of size-exclusion chromatography.

12. A peptide product obtained according to any of the preceding claims with purity ranging from 97-100%.

13. A peptide product obtained according to any of the preceding claims with a purity of atleast 96%.

14. A method of purifying a cyclic or non-cyclic peptide and related analogs or derivatives from a mixture containing at least one related impurity through reverse-phase chromatography comprising steps of:
a) packing the RP-HPLC column with a silica based polymer resin equilibrated with about 5% of a polar solvent in an organic acid buffer;
b) loading the peptide composition containing atleast one related impurity on the column at a flow rate of about ≦100-400 cm/hr;
c) washing the column with the same buffer solution as in Step a; and
d) eluting the purified product from the column performing a linear gradient of 8-14%.

15. A method of purifying a cyclic or non-cyclic peptide and related analogs or derivatives from a mixture containing at least one related impurity through ion-exchange chromatography comprising steps of:
a) equilibrating the cation-exchange column with an aqueous solution of a weak acid buffer;
b) loading the RP-HPLC purified peptide onto the column; and
c) washing the column and eluting the peptide product with the buffer as used in step a;

16. A method of purifying a cyclic or non-cyclic peptide and related analogs or derivatives from a mixture containing at least one related impurity contacting the said peptide mixture in any sequence of order, with a RP-HPLC chromatographic matrix and/or ion-exchange chromatographic matrix said purification process comprising steps of:
a) packing the RP-HPLC column with a silica based polymer resin equilibrated with about 5% of a polar solvent in an organic acid buffer;
b) loading the peptide composition containing atleast one related impurity on the column at a flow rate of about ≦100-400 cm/hr;
c) washing the column with the same buffer solution as in Step a;
d) eluting the purified product from the column performing a linear gradient of 8-14%;
e) equilibrating the cation-exchange column with an aqueous solution of a weak acid buffer;
f) loading the RP-HPLC purified peptide onto the column; and
g) washing the column and eluting the peptide product in elution buffer

17. The method according to claim 14, 15 or 16 wherein the resin may be selected from the group comprising Sephadex, methylacrylate resin, carboxymethyl cellulose, carboxymethyl sephadex, sulphopropyl cellulose, sulphopropyl sephadex.

18. The method according to claim 14, 15 or 16, wherein the resin is polystyrene or divinylbenzene.

19. The method according to claim 14, 15 or 16, wherein the resin bead has a particle size of 1-50 μm.

20. The method according to claim 14, 15 or 16, wherein the pore size of the resin bead is 100-500 Å.

21. The method according to claim 14, 15 or 16, wherein said method further comprising another optional step of size-exclusion chromatography.

22. The method according to claim 14, 15 or 16, wherein the cyclic or non-cyclic peptide is selected from the group comprising eptifibatide, exenatide, atosiban or nesiritide.

23. The method according to claim 14, 15 or 16, wherein the polar buffer solvent is acetonitrile.

24. The method according to claim 14, 15 or 16, wherein the organic acid buffer is selected from the group comprising citric acid, acetic acid, perchloric acid and formic acid.

25. The method according to claim 2, wherein the molarity of the buffer used is ranging from 10 mM-50 mM.

26. The method according to any of the preceding claims 14 to 25, wherein the purification is carried out at a pH ranging from 2-9.

27. The peptide product obtained according to any of the preceding claims 14 to 26, wherein the purity is atleast 96%.

28. The peptide product obtained according to any of the preceding claims 14 to 26 with purity ranging from 97-100%.

29. Purified eptifibatide with purity of atleast 96%.

30. Purified exenatide with purity of atleast 96%.

31. Purified atosiban with purity of atleast 96%.

32. Purified nesiritide with purity of atleast 96%.

33. A method for purifying a peptide and purified peptide product are substantially as herein described along with accompanying drawings and examples.
